(19) United States
(12) Patent Application Publication
(10) Pub. No.: US 2018/0353208 A1
(43) Pub. Date: Dec. 13, 2018

(54) COLLECTION APPARATUS

(71) Applicant: PRK LIMITED, Hong Kong (CN)

(72) Inventor: Christopher Schenk, Austin, TX (US)

(21) Appl. No.: 15/553,731

(22) PCT Filed: Feb. 25, 2015
(86) PCT No.: PCT/US2015/017566

§ 371 Date: Aug. 25, 2017

(51) Int. Cl. A61B 17/43

(52) U.S. Cl. CPC A61B 17/43

(57) ABSTRACT

A sperm collection apparatus includes a housing defining an interior chamber and having a proximal opening at a proximal end of the housing and a distal opening at a distal end of the housing. An inner sleeve includes a gel-like material and is disposed within the housing. The inner sleeve defines an internal insertion channel that is sized for closely receiving a user's penis and has an insertion opening at the proximal opening of the housing.

### BACKGROUND

The present invention relates to a sperm collecting apparatus. In particular, the present invention relates to such an apparatus having improved features including a powder vessel for introducing powder into the apparatus to aid in drying and restoring the inner sleeve, an air release valve, and removable rings for changing the size of a the insertion opening.

Sperm collection devices have been previously known. Examples of such devices can be seen in U.S. Pat. Nos. 5,807,360, 7,753,895 and 7,993,315. Typically, such devices have a gel-like inner sleeve disposed within a housing and defining an insertion opening and channel for receiving a user's penis.

It is an object of the present invention to provide an improved sperm collection apparatus that can be easily cleaned without removing the inner sleeve and can be relatively easily dried and restored with powder after cleaning.

It is another object of the present invention to provide an improved sperm collection apparatus that includes an air release valve for controlling the air pressure within the apparatus during use.

It is another object of the present invention to provide an improved sperm collection apparatus that allows the user to change the size of the insertion opening.

Additional objectives and advantages of the invention will be set forth in the description that follows, and in part will be apparent from the description, or may be learned by practice of the invention. The objects and advantages of the invention may be realized and obtained by means of the instrumentalities and combinations pointed out in this specification and the appended claims.

### SUMMARY

To achieve the foregoing objectives, and in accordance with the purposes of the invention as embodied and broadly described in this document, there is provided a sperm collection apparatus including a housing defining an interior chamber and having a proximal opening at a proximal end of the housing and a distal opening at a distal end of the housing. In some embodiments, the housing can include a rubberized coating on its exterior surface. The proximal opening and the distal opening are in communication with the interior chamber. A proximal removable cap is adapted for covering the proximal opening. An inner sleeve includes a gel-like material and is disposed within the housing. The inner sleeve defines an internal insertion channel that is sized for closely receiving a human penis and has an insertion opening at the proximal opening of the housing. A powder vessel is sized to fit within the removable cap and is adapted for collecting powder introduced into the housing by depositing powder into the powder vessel, shaking the housing in order to distribute the powder within the inner sleeve and thereafter, collecting powder in the powder vessel. The collection apparatus can include a distal removable cap for covering the distal opening. A grating can be provided at the distal opening, wherein water or another suitable fluid can be introduced into the housing through the grating, or through the proximal opening of the inner sleeve's insertion channel, for rinsing the inner sleeve prior to drying with powder.

In some embodiments, the apparatus can include a valve for releasing air from the insertion channel during use. The valve can be disposed near the distal end of the housing. In some embodiments, the valve is disposed in the distal cap and is in fluid communication with the inner sleeve insertion channel. The distal cap can include a lever for opening and closing the valve.

In some embodiments, the apparatus includes a stopper adapted to closely fit the distal opening. The stopper has an opening in communication with the inner sleeve insertion channel and a mating portion for mating with the cap for covering the stopper opening. The apparatus can include a stopper seal adapted to provide a substantially air-tight seal between the stopper and the distal opening of the housing. The apparatus also can include a cap seal adapted to provide a substantially air-tight seal between and the stopper and the removable cap when the stopper mating portion is mated with the removable cap.

In some embodiments, the collecting apparatus can include one or more removable rings adapted to be disposed near the housing proximal opening to provide an inwardly depending flange for constricting the insertion channel opening of the inner sleeve. Each of the rings can have a different inner diameter for constricting the insertion channel opening to a different degree.

In some embodiments, one or more projections are disposed on the housing and adapted to hold the inner sleeve in a fixed relationship with the housing.

### BRIEF DESCRIPTION OF THE DRAWINGS

The accompanying drawings, which are incorporated in and constitute a part of the specification, illustrate the presently preferred embodiments and methods of the invention and, together with the general description given above and the detailed description of the preferred embodiments and methods given below, serve to explain the principles of the invention.

FIG. 1 is a front elevation view of one embodiment of a sperm collecting apparatus according to the present invention, with the proximal and distal caps on.

FIG. 2 is an exploded view of the apparatus of FIG. 1, showing among other elements of the apparatus, the housing, the distal cap assembly with the valve opening, the distal opening stopper, the inner sleeve, the constricting ring, and the proximal cap assembly with the powder vessel.

FIG. 3 is an elevation view of the housing of the apparatus of FIG. 1.

FIG. 4 is a bottom plan view of the housing of FIG. 3.

FIG. 5 is a side elevation view of the distal opening stopper of the apparatus of FIG. 1.

FIG. 6 is a top plan view of the distal opening stopper of FIG. 5.

FIG. 7 is a top plan view of the proximal cap of the apparatus of FIG. 1.

FIG. 8 is a bottom plan view of the proximal cap of the apparatus of FIG. 1.

FIG. 9 is a perspective view of the proximal cap assembly of the apparatus of FIG. 1 showing the powder vessel inserted into the cap.

FIG. 10 is a top plan view of the powder vessel of the apparatus of FIG. 1.

FIG. 11 is a side elevation view of the powder vessel of the apparatus of FIG. 1.

FIG. 12 is a side elevation view of the distal cap assembly of the apparatus of FIG. 1.

FIG. 13 is a top plan view of the distal cap assembly of FIG. 12.

FIG. 14 is a top plan view of the distal cap assembly of FIG. 12, showing the cap lid removed.

FIG. 15 is a bottom plan view of the distal cap assembly of FIG. 12.

FIG. 16 is a top plan view of the lid of the distal cap assembly of FIG. 12.

FIG. 17 is a side elevation view of the lid of the distal cap assembly of FIG. 12.

FIG. 18 is a perspective view of the distal cap assembly of FIG. 12, showing the air control valve in the closed position.

FIG. 19 is a perspective view of the distal cap assembly of FIG. 12, showing the air control valve being opened by pressing lid of the distal cap assembly.

FIG. 20 is a perspective view of the distal end of the housing showing how the stopper is attached to the housing.

FIG. 21 is a partial cutaway view of the housing and the inner sleeve of the apparatus of FIG. 1, showing the inner sleeve disposed within the housing.

FIG. 22 is a perspective view of the inner sleeve of the apparatus of FIG. 1.

FIG. 23 is a partial cutaway view of the inner sleeve of FIG. 22.

FIG. 24 is a cross-sectional view of the inner sleeve of FIG. 22.

### DESCRIPTION

Reference in this application is made to presently preferred embodiments of the invention. While the invention is described more fully with reference to these examples, the invention in its broader aspects is not limited to the specific details, representative devices, and illustrative examples shown and described. Rather, the description is to be understood as a broad, teaching disclosure directed to persons of ordinary skill in the appropriate arts, and not as limiting upon the invention.

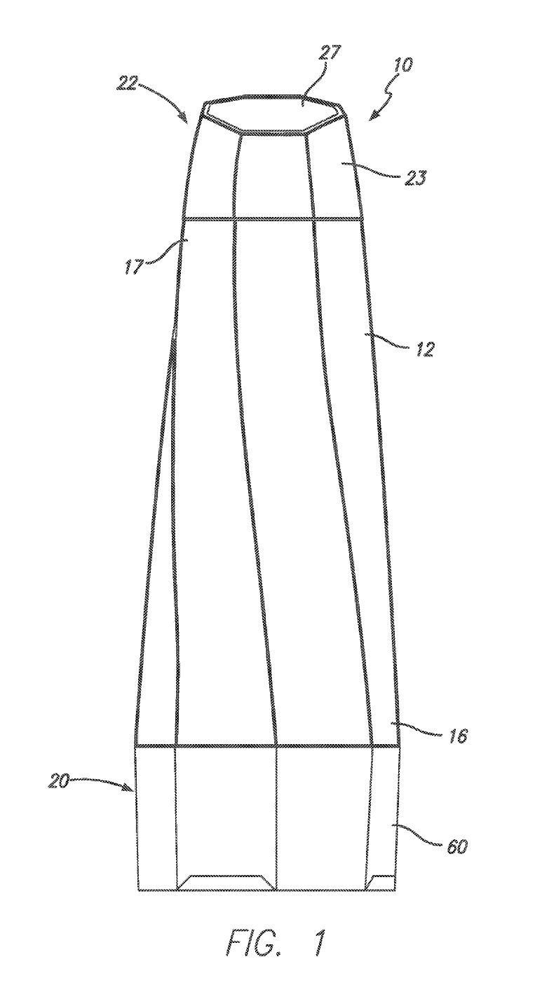

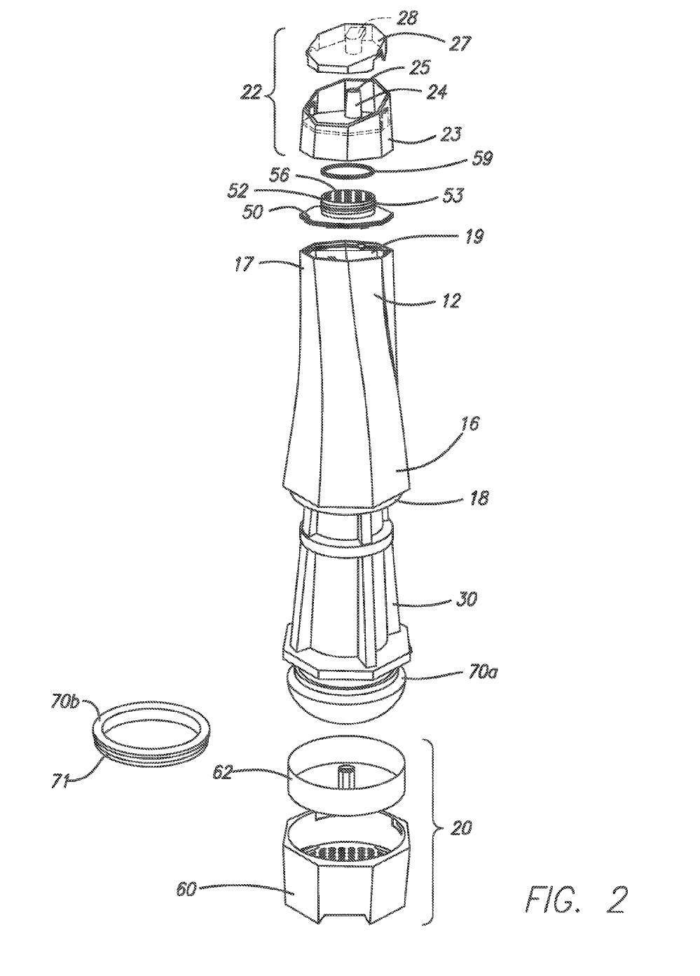

Referring generally to FIGS. 1-2, a presently preferred embodiment of a sperm collection apparatus 10 according to the invention includes a container having a generally cylindrical housing 12 defining an interior chamber 14 (sec FIG. 14) and having a proximal end 16 and a distal end 17. In one embodiment, the housing 12 is provided with a rubberized coating on the housing exterior surface, which can be applied to the housing by spraying the exterior surface of the housing with a polyurethane-based, rubberized liquid paint, and thermally treating the paint by “baking it on” immediately following its application to the exterior surface of the housing. One example of such a paint is Soft Touch Coating Paint, which can be obtained from Forever Coatings Limited of Hong Kong.

Still referring to FIGS. 1-2, an inner sleeve 30 is disposed inside the housing 12 and is made from a gel-like material, as is known in the art. The inner sleeve 30 defines an internal elongated insertion channel 34 (as can be seen in FIG. 21) that is sized for closely receiving a user's penis. The housing proximal end 16 has an opening 18 that is in communication with the housing interior chamber 14. The housing distal end 17 has an opening 19 that also is in communication with the housing interior chamber 14 and is adapted for attaching a distal cap assembly 22 to cover the housing distal opening 19. Near the housing proximal opening 18, the inner sleeve 30 defines an insertion opening 32 that is in communication the insertion channel 34, as can be seen in FIG. 21.

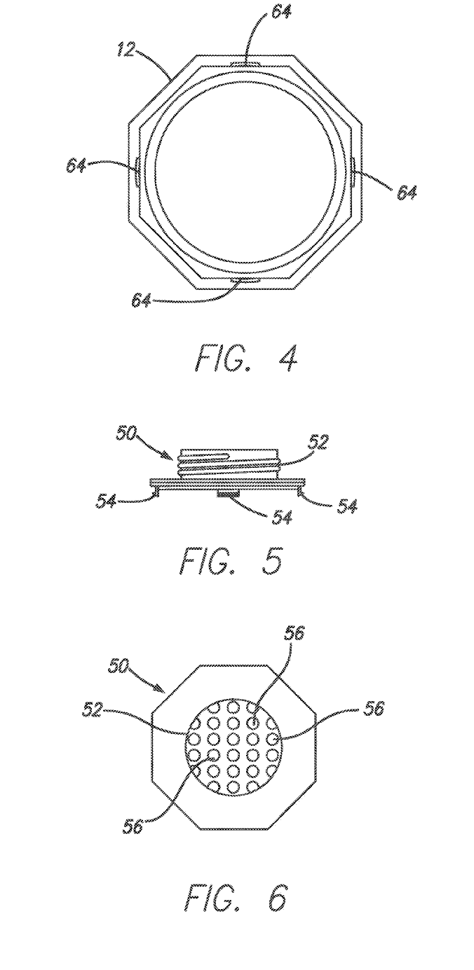

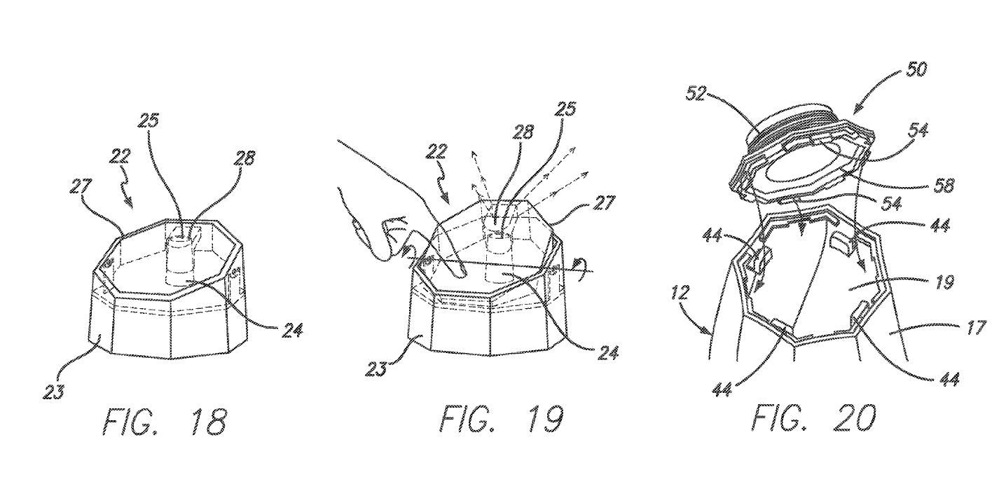

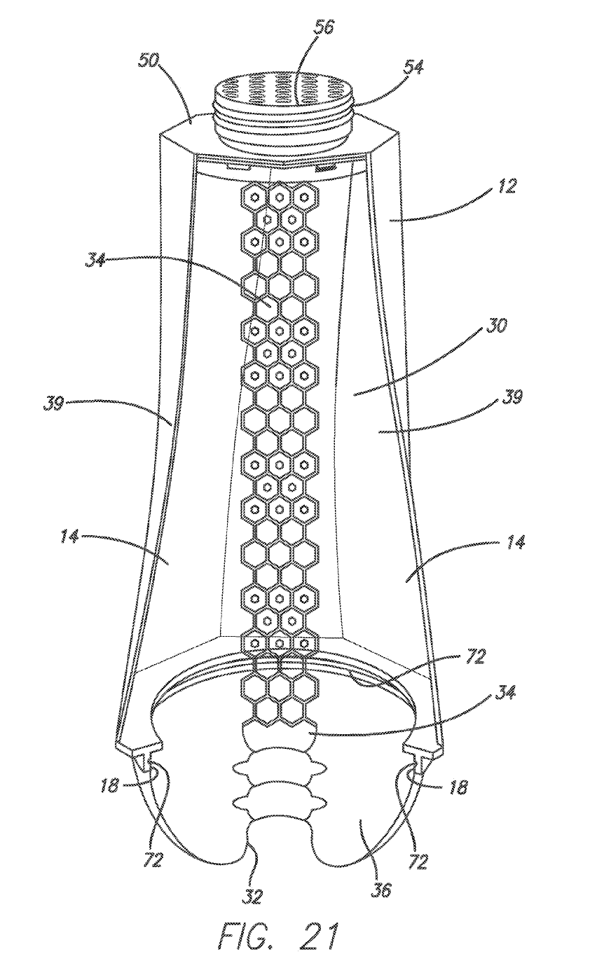

Referring to FIGS. 2, 5-6, 20 and 21, the apparatus 10 includes a stopper 50 adapted to closely fit the distal opening 19. The stopper 50 has a mating portion 52 that includes threads 53 for mating with the distal cap assembly 22. Clips 54 are disposed on a bottom rim of the stopper 50 for attaching it to the housing distal end 17 to cover the housing distal opening 19. Openings in the form of a grate 56 in the top of the stopper 50 are in communication with the insertion channel 34 of the inner sleeve 30 when the stopper 50 is attached to the housing distal end 17. A stopper bottom seal 58 is positioned around on the bottom rim of the stopper 50 (see FIG. 20) to provide a substantially air-tight seal and prevent air leakage at the joint between the stopper 50 and the housing distal end 17. In one embodiment, the stopper bottom seal 58 is molded from silicone in a shape that includes openings through which the stopper clips 54 project, so that the stopper bottom seal 58 can provide an air-tight seal around the clips 54 when the stopper 50 is attached to the housing distal end 17. A stopper top seal 59 (see FIG. 2) is positioned within the distal cap assembly 22 to provide a substantially air-tight seal and prevent air leakage at the joint between the stopper 50 and the distal cap assembly 22 when the distal cap assembly 22 is screwed on to the stopper mating portion 52. In one embodiment, the stopper top seal 59 is molded from silicone in a shape that lines the joint between the stopper 50 and the distal cap assembly 22.

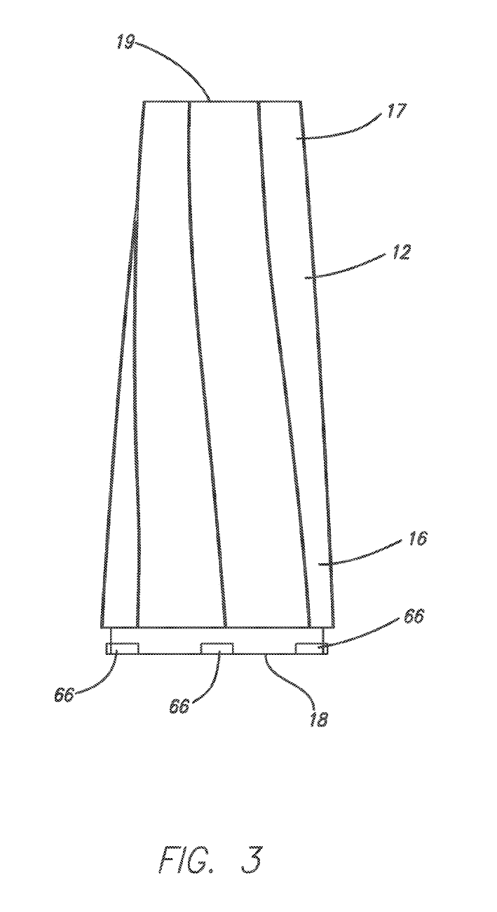

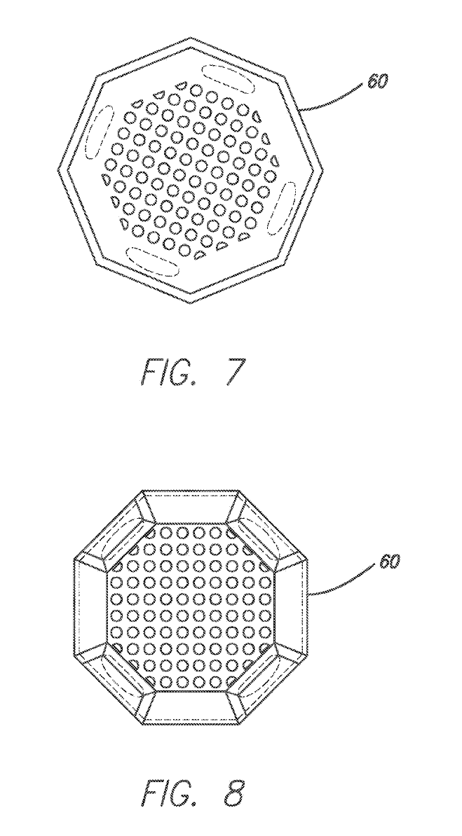

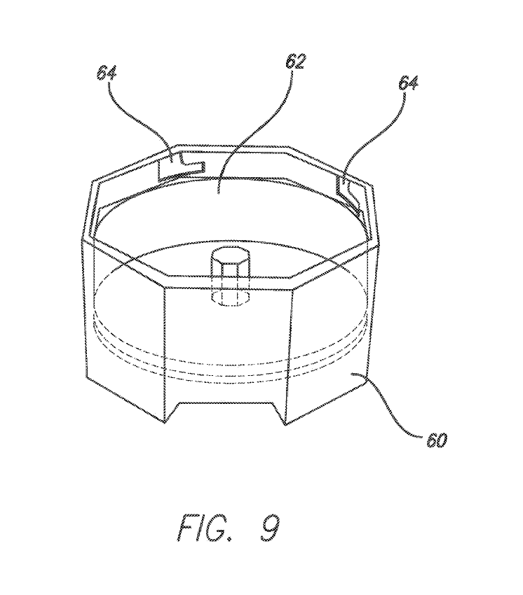

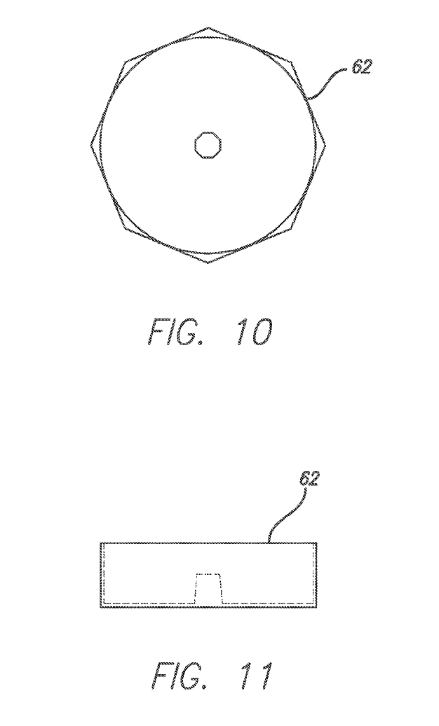

Referring to FIGS. 1-9, a proximal cap assembly 20 is adapted to be removably attached to the housing proximal end 16 to cover the housing proximal opening 18. The proximal cap assembly 20 includes a removable proximal cap 60 and a powder vessel 62 sized to fit within the proximal cap 60. The purpose of the powder vessel 62 is to introduce powder into the housing 12 to aid in drying and restoring the inner sleeve 30 and to then collect the residual powder. Powder is introduced into the housing 12 by depositing the powder into the powder vessel 62, shaking the assembled apparatus 10 in order to distribute the powder within the inner sleeve 30, and, thereafter, collecting any residual powder in the powder vessel 62. The proximal cap 60 includes slots 64 that receive and mate with corresponding tabs 66 on the housing proximal end 16 and thereby allow for removable attachment (via twisting) to the housing proximal end 16 to cover the housing proximal opening 18 and the inner sleeve proximal end 36 when the apparatus 10 is not in use.

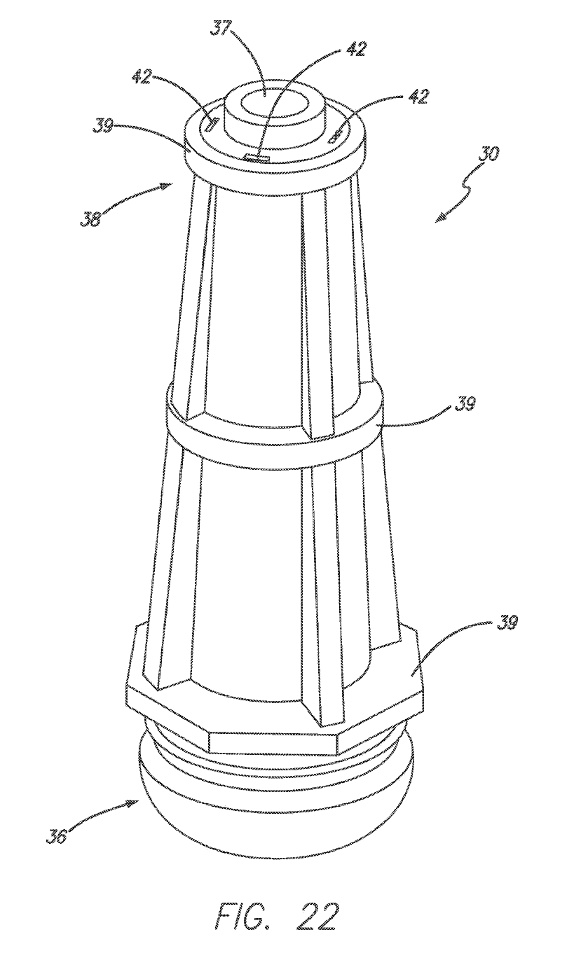

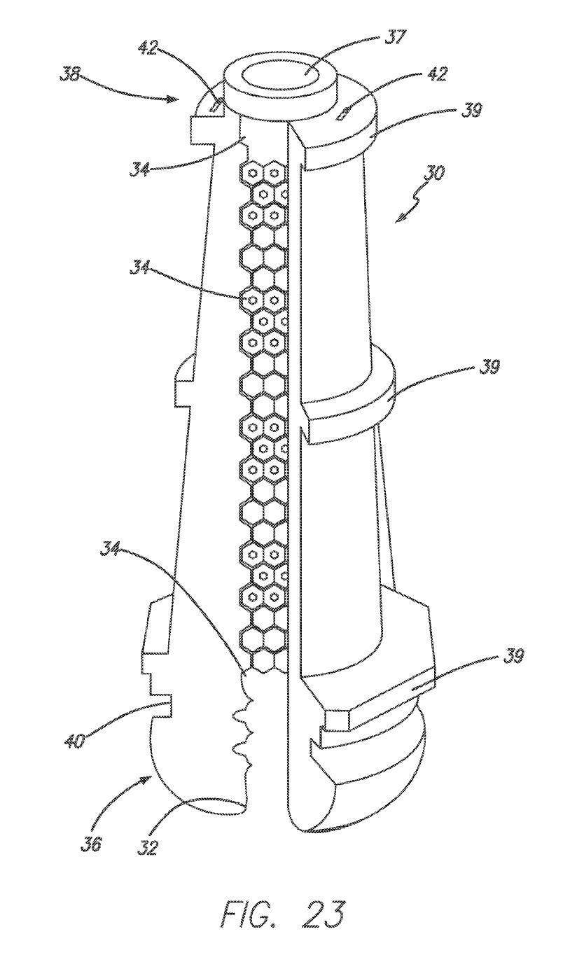

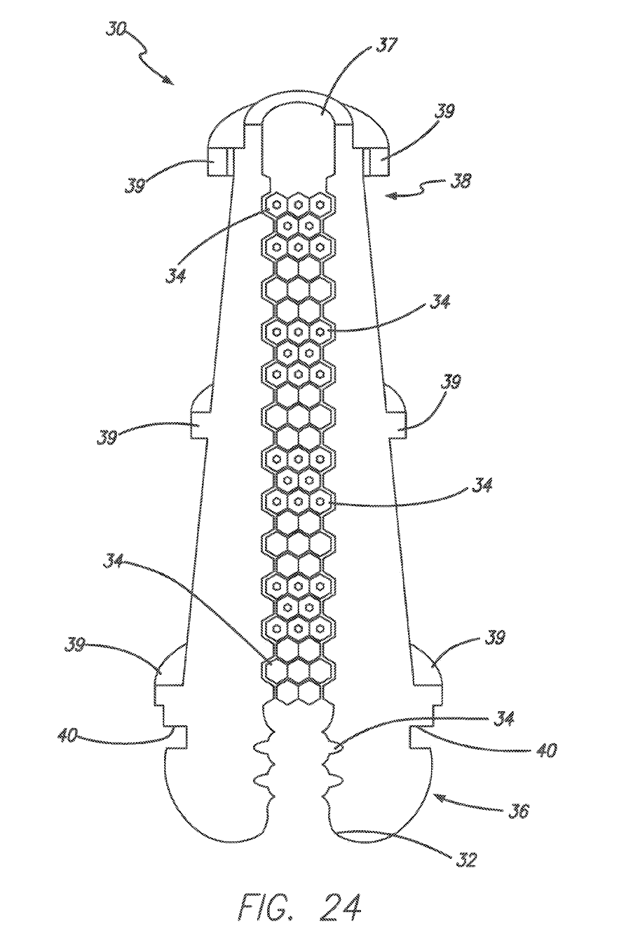

Referring to FIGS. 2 and 21-24, the inner sleeve 30 has a proximal end 36 and a distal end 38 and is of a size and shape to fit within the housing interior chamber 14. The inner sleeve 30 is made of a gel-like substance, as is known in the art. The inner sleeve 30 defines an internal insertion channel 34 that extends the length of the inner sleeve 14 from the insertion opening 32 at a proximal end 36 to a distal opening 37 at an inner sleeve distal end 38. The insertion channel 34 can be configured with varying diameters, geometries, projections, ridges and folds along its length to provide a user with the sensation of a life-like canal and with enhanced sensations during use. The inner sleeve 30 has external spacing ribs 39 for contacting the wall of the housing interior chamber 14 and holding the inner sleeve 30 in spaced relationship to that wall. At the inner sleeve proximal end 36 a peripheral channel 40 is sized to receive a removable ring 70 as described below. At the inner sleeve distal end 38, slots 40 are provided for receiving hooked projections 44 disposed on the wall of the housing interior chamber 14 near the housing distal opening 19 to hold the inner sleeve 30 in a fixed relationship within the housing 12 during use. During assembly of the apparatus 10, the inner sleeve 30 is inserted into the housing interior chamber 14 through the housing proximal opening 18 and the slots 40 are stretched over the hooked projections 44 to securely hold the inner sleeve proximal end 36 in place.

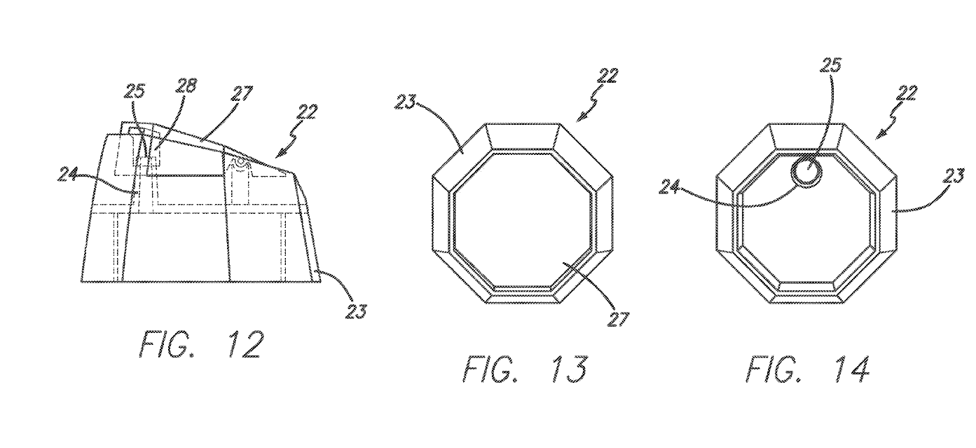

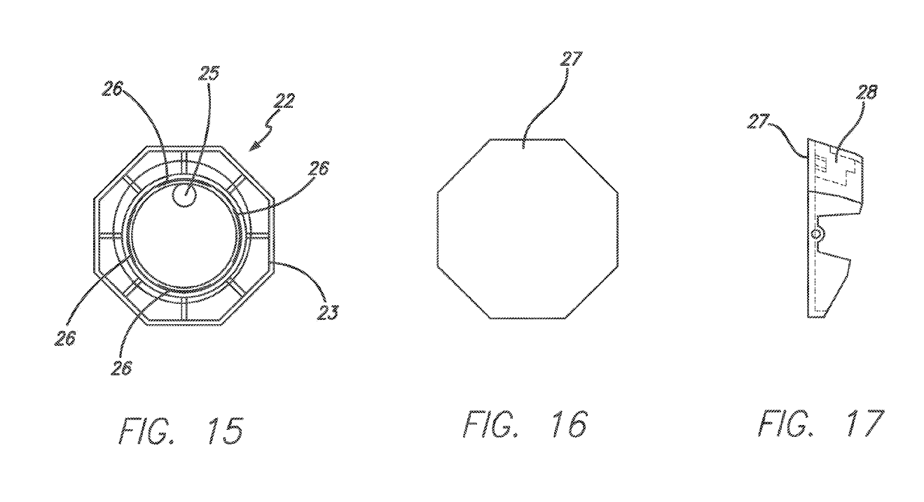

Referring to FIGS. 1-2 and 13-19, the collection apparatus 10 includes a distal cap assembly 22 for covering the stopper 50. The distal cap assembly 22 includes a distal cap body 23 having internal threads 26 that mate with the threads 54 of the stopper 50 so that the distal cap body 23 can be removably mounted to the housing distal end 17 by screwing the distal cap body 23 to the stopper 50. The distal cap body 23 includes a vent tube 24 having a vent opening 25 in communication with the interior of the distal body cap 23, which in turn is in fluid communication with the inner sleeve insertion channel 34. A distal cap lid 27 is pivotally mounted to the distal cap body 23. A vent tube cap 28 is disposed on the bottom of the cap lid 27 and is sized to cover the vent opening 25. When the lid 27 is rotated into a closed position, as shown FIG. 18, the vent tube cap 28 moves downward thereby closing off the vent opening 25 to air flow. When the lid 27 is rotated into an open position, as shown FIG. 19, the vent tube cap 28 is moves upward thereby and allows air to flow through the vent opening 25. In this configuration, the lid 27 acts as a lever with which a user can open and close the vent opening 25 to vary pressure difference between an inner pressure and the atmospheric pressure and thereby vary the tightness of the contact between the inner sleeve 30 and the user's penis during use.

Referring again to FIGS. 2 and 21-24, the collecting apparatus 10 can include a removable ring 70 adapted to be disposed near the housing proximal opening 18 to provide an inwardly depending flange for constricting the insertion channel 34 of the inner sleeve 30 near the insertion opening 32. Alternate rings 70a, 70b, having different inner diameters, can be used for constricting the insertion channel 34 to different degrees. Each removable ring 70 has an outer peripheral lip 71 (see FIG. 2) that nests into a corresponding peripheral recess 72 disposed around the interior of the housing proximal end (see FIG. 21) when the ring 70 is snapped into place. To replace one ring 70a with another 70b, a user need only push the inner sleeve proximal end 36 through the ring 70a into the housing proximal end 16, pop out the ring 70a, snap in the other ring 70b into the housing proximal end 16, and pull the inner sleeve proximal end 36 out of the housing proximal end 16 until it is back in place.

Before using the apparatus 10, a user can optionally warm the inner sleeve 30 by removing the distal cap assembly 22 and running warm water through the inner sleeve 30 for a few minutes to allow sleeve material to absorb the heat. This step is optional, but makes for a more realistic experience when using the apparatus 10. After warming the inner sleeve 30 in this manner, the use replaces the distal cap assembly 22. After the user inserts his penis into the apparatus 10, he can control the suction provided by the apparatus 10 by simply pressing the cap lid 27 so that the vent opening 25 on the distal cap assembly is open or closed during use.

After use, the apparatus 10 can be cleaned by unscrewing and removing the distal cap assembly 22 and then rinsing out the inner sleeve 30 with warm water or another suitable cleaning fluid. To dry the apparatus 10, it can be set on its ventilated proximal cap 60 (with the powder cup 62 removed). The user can press the distal cap lid 27 into the open position to allow the inner sleeve 30 to dry naturally and discreetly. For quicker drying time, the user can unscrew and remove the distal cap assembly 22 completely. After the inner sleeve 30 is partly dry, but remains damp, powder can be introduced into the inner sleeve 30 to help completely dry/absorb any small amounts of remaining liquid. In addition to drying the inner sleeve 30, the powder can help to restore the inner sleeve 30 to its original feel. With the presently preferred embodiment, powder is introduced into the inner sleeve 30 by placing the powder vessel 62 into the proximal cap 60 pouring a suitable amount of powder into the powder vessel 62, and attaching the proximal cap 60 to the housing proximal end 16 and locking it closed (via twisting). The user can then shake the assembled apparatus 10 to introduce the powder into the inner sleeve 30.

Having read this disclosure, it will also be understood by those having skill in the art that modifications may be made to the invention without departing from its spirit and scope. Therefore, the invention in its broader aspects is not limited to the specific details, representative devices, and illustrative examples shown and described. Accordingly, departures may be made from such details without departing from the spirit or scope of the general inventive concept.

What is claimed is:

1. A sperm collecting apparatus comprising:
a housing defining an interior chamber and having a proximal opening at a proximal end of the housing and a distal opening at a distal end of the housing, wherein the proximal opening and the distal opening are in communication with the interior chamber;
a proximal removable cap for covering the proximal opening;
an inner sleeve comprising a gel-like material and disposed within the housing, wherein the inner sleeve defines an internal insertion channel that is sized for closely receiving a human penis and has an insertion opening at the proximal opening of the housing; and
a vessel sized to fit within the removable cap and adapted for collecting a powder introduced into the inner sleeve for drying or restoring the inner sleeve.

2. The sperm collecting apparatus of claim 1 further comprising a distal removable cap for covering the distal opening.

3. The sperm collecting apparatus of claim 1 further comprising a grating at the distal opening, wherein a fluid can be introduced into or released from the housing through the grating.

4. The sperm collecting apparatus of claim 1 further comprising a valve at the distal end of the housing in communication with the interior chamber for releasing air.

5. The sperm collecting apparatus of claim 4 wherein the valve comprises a lever for opening and closing the valve.

6. The sperm collecting apparatus of claim 1 further comprising a removable flange disposed at the proximal end, wherein the flange constricts the insertion channel opening of the inner sleeve.

7. The sperm collecting apparatus of claim 1 wherein the housing includes a rubberized coating on a housing exterior surface.

8. A sperm collecting apparatus comprising:
a housing defining an interior chamber and having a proximal opening at a proximal end of the housing and a distal opening at a distal end of the housing, wherein the proximal opening and the distal opening are in communication with the interior clumber;
an inner sleeve comprising a gel-like material and disposed within the housing, wherein the inner sleeve defines an internal insertion channel that is sized for closely receiving a human penis and has an insertion opening at the proximal opening of the housing; and
one or more removable flanges adapted to be disposed at the proximal end, and to constrict the insertion channel opening of the inner sleeve.

9. The sperm collecting apparatus of claim 8 further wherein the one or more removable flanges comprises a plurality of flanges and each of the plurality of flanges has a different inner diameter for constricting the insertion channel opening.

10. The sperm collecting apparatus of claim 8 further comprising a proximal removable cap for covering the proximal opening.

11. The sperm collecting apparatus of claim 8 further comprising a distal removable cap for covering the distal opening.

12. The sperm collecting apparatus of claim 8 further comprising a grating at the distal opening, wherein a fluid can be introduced into or released from the housing through the grating.

13. The sperm collecting apparatus of claim 8 further comprising a valve at the distal end of the housing in communication with the interior chamber for releasing air.

14. The sperm collecting apparatus of claim 13 wherein the valve comprises a lever for opening and closing the valve.

15. The sperm collecting apparatus of claim 8 wherein the housing includes a rubberized coating on a housing exterior surface.

16. A sperm collecting apparatus comprising:
a housing defining an interior chamber and having a proximal opening at a proximal end of the housing and a distal opening at a distal end of the housing, wherein the proximal opening and the distal opening are in communication with the interior chamber;
an inner sleeve comprising a gel-like material and disposed within the housing, wherein the inner sleeve defines an internal insertion channel that is sized for closely receiving a human penis and has an insertion opening at the proximal opening of the housing; and
a valve disposed in the housing in communication with the interior chamber for releasing air.

17. The sperm collecting apparatus of claim 16 wherein the valve is disposed at the distal end of the housing.

18. The sperm collecting apparatus of claim 16 wherein the valve comprises a lever for opening and closing the valve.

19. The sperm collecting apparatus of claim 16 further comprising a distal removable cap for covering the distal opening, wherein the valve is disposed in the distal cap.

20. The sperm collecting apparatus of claim 16 further comprising a grating at the distal opening, wherein a fluid can be introduced into or released from the housing through the grating.

21. The sperm collecting apparatus of claim 16 further comprising a removable flange disposed at the proximal end and adapted for constricting the insertion channel opening of the inner sleeve.

22. The sperm collecting apparatus of claim 16 wherein the housing includes a rubberized coating on a housing exterior surface.

23. The sperm collecting apparatus of claim 16 further comprising a powder vessel adapted for collecting a powder introduced into the housing for drying or restoring the inner sleeve.

24. The sperm collecting apparatus of claim 23 wherein the powder vessel is disposed within a removable cap for covering the proximal end of the housing.

25. A sperm collecting apparatus comprising:
a housing defining an interior chamber and having a proximal opening at a proximal end of the housing and a distal opening at a distal end of the housing, wherein the proximal opening and the distal opening are in communication with the interior chamber;
a proximal removable cap for covering the proximal opening;
an inner sleeve comprising a gel-like material and disposed within the housing, wherein the inner sleeve defines an internal insertion channel that is sized for closely receiving a human penis and has an insertion opening at the proximal opening of the housing; and
a grating adapted for allowing a powder or a fluid to be introduced into the housing through the grating for drying or restoring the inner sleeve.

26. A sperm collecting apparatus comprising:
a housing defining an interior chamber and having a proximal opening at a proximal end of the housing and a distal opening at a distal end of the housing, wherein the proximal opening and the distal opening are in communication with the interior chamber;
a proximal removable cap for covering the proximal opening; and
an inner sleeve comprising a gel-like material and disposed within the housing, wherein the inner sleeve defines an internal insertion channel that is sized for closely receiving a human penis and has an insertion opening at the proximal opening of the housing;
wherein the housing includes a rubberized coating on a housing exterior surface.

27. A sperm collecting apparatus comprising:
a housing defining an interior chamber and having a proximal opening at a proximal end of the housing and a distal opening at a distal end of the housing, wherein the proximal opening and the distal opening are in communication with the interior chamber;
an inner sleeve comprising a gel-like material and disposed within the housing, wherein the inner sleeve defines an internal insertion channel that is sized for closely receiving a human penis and has an insertion opening at the proximal opening of the housing;
a stopper adapted to closely fit the distal opening, wherein the stopper includes an opening in communication with the interior chamber and a mating portion for mating with a removable cap for covering the stopper opening; and
a stopper seal adapted to provide a substantially air-tight seal between the stopper and the distal opening of the housing.

28. The sperm collecting apparatus of claim 27 further comprising a cap seal adapted to provide a substantially air-tight seal between and the stopper and the removable cap when the stopper mating portion is mated with the removable cap.

29. A sperm collecting apparatus comprising:
a housing defining an interior chamber and having a proximal opening at a proximal end of the housing and a distal opening at a distal end of the housing, wherein the proximal opening and the distal opening are in communication with the interior chamber;
an inner sleeve comprising a gel-like material and disposed within the housing, wherein the inner sleeve defines an internal insertion channel that is sized for closely receiving a human penis and has an insertion opening at the proximal opening of the housing; and
one or more projections disposed on the housing and adapted to hold the inner sleeve in a fixed relationship with the housing.

30. The sperm collecting apparatus of claim 29 wherein the at least one of the one or more projections is disposed near the distal opening of the housing.
